(19) United States
(12) Patent Application Publication
(10) Pub. No.: US 2025/0108001 A1
(43) Pub. Date: Apr. 3, 2025

(54) TOPICAL OCULAR ADMINISTRATION OF AMBROXOL FOR OCULAR PAIN

(71) Applicant: NeuVision Development LLC, Tucson, AZ (US)

(72) Inventors: Mingwu Wang, Tucson, AZ (US); Cindy A. Wang, Tucson, AZ (US)

(21) Appl. No.: 18/865,673

(22) PCT Filed: May 19, 2023
(86) PCT No.: PCT/US2023/067268

§ 371 Date: Nov. 13, 2024

Related U.S. Application Data
(60) Provisional application No. 63/344,561, filed on May 21, 2022.

(51) Int. Cl. A61K 9/00; A61K 31/137; A61K 31/167; A61K 31/245; A61K 31/4453; A61K 45/06; A61K 47/40; A61P 25/04

(52) U.S. Cl. CPC A61K 9/0048; A61K 31/137; A61K 31/167; A61K 31/245; A61K 31/4453; A61K 45/06; A61K 47/40; A61P 25/04

(57) ABSTRACT

Methods and formulations for treating ocular pain, including nociceptive and neuropathic ocular pain, are disclosed. The formulations include ambroxol or a chemical derivative thereof. The methods and formulations selectively inhibit the Nav1.8 nociceptor which allow sensation while relieving pain. The formulation and method provide effective and safer topical ocular pain control than current topical analgesic formulations and methods.

### CROSS-REFERENCE TO RELATED APPLICATIONS

This application claims the benefit of and priority to U.S. Application No. 63/344,561 filed on May 21, 2022, the content of which is incorporated herein by reference in its entirety.

### BACKGROUND

The sensory systems of touch and pain provide us with information about our environment and our bodies that is often crucial for survival and well-being. Sensation of such information about our environment and our bodies is coded in the periphery and interpreted by the brain as touch and pain sensations. When well-functioning, these circuits promote survival and prepare us to make adaptive decisions. Pathological loss of touch can result in perceived disconnection from the body, and insensitivity to pain can be dangerous, leading to maladaptive hazardous behavior. Pain from tissue inflammation or injury can adversely affect quality of life of patients even though in such circumstances pain sensation serves as a protective mechanism of our bodies. On the other hand, conditions in which these systems start signaling pain in response to innocuous touch or even in the absence of any observable sensory stimuli, have tremendous negative impact on the lives of the affected as well.

Our pain system at the ocular surface (composed essentially of the cornea and conjunctiva) is unique in its principal role of protecting, sustaining, and restoring the optical tear layer without which our vision would be non-functional. To meet this existential need, the neuronal network of the cornea has evolved into the most powerful, sensitive, and complex pain system in the body, incorporating sensors that are unusually vulnerable to being exposed to the noxious environment. The cornea has the highest density of nociceptors of any tissues in the body and maintains an elevated level of sensitivity even to normal environmental stimuli. Hence, ocular pain derives mostly from the ocular surface tissues, predominantly from the cornea. As such, experimentations on ocular pain center at the ocular surface, predominantly at the cornea. Due to the exclusively unique, highly functionally sensitive, and structurally complex neurosensory system at the cornea, pain medications effective in other parts of the body may not be effective or safe when used for ocular pain control.

For example, topical 0.1% diclofenac, a non-steroidal anti-inflammatory drug (NSAID), does not have clinically significant cornea anesthetic effect in humans. Commonly used topical ophthalmic anesthetic drugs include but are not limited to tetracaine, proparacaine, lidocaine, and bupivacaine. Continuous topical application of anesthetics or NSAIDs to the eye is known to increase the incidence of infections and corneal scarring and delay corneal wound healing in patients with epithelial defects; long-term use can increase the risk of corneal melts and even perforation. Novel pain management strategies with fewer side effects are needed to alleviate ocular pain.

In contrast to physiological nociceptive ocular pain, neuropathic ocular pain (NOP) on the other hand is a spectrum of disorders of ocular pain caused by damage or diseases affecting nervous tissue, but the condition can also be caused by peripheral or centralized sensitization. NOP is frequently seen in illnesses associated with ocular surface tissue damage such as infections, trauma, various ocular surgeries including laser refractive procedures, and chronic severe ocular surface destructions such as limbal stem cell deficiency and sequela of chemical burn, etc. When caused by peripheral or centralized sensitization, NOP patients may have little or no signs of ocular surface tissue damage, and frequently respond poorly to conventional analgesic treatments. These patients may also have pain symptoms that are out of proportion to the presentation of their clinical diseases.

The experience of painful sensations in NOP can vary widely, reflecting a variety of causal factors such as: types of noxious stimuli causing insult to ocular surface nociceptors, the types of corneal sensory receptors affected, the extent of the inflammatory responses, and the type or types of disorders and damage affecting the nervous system. Symptoms of NOP can range from devastating, unrelenting eye pain and severe sensitivity to light (photophobia) in the worst cases, to mild hyperalgesia (increased sensitivity to feeling pain and an extreme response to pain) or dysesthesia (distorted sense, especially touch).

Distorted sensations and levels of pain can vary depending on the source(s) of the maladaptive signals (e.g., abnormal axonal regeneration, or peripheral sensitization). One or both eyes may be affected, with varying degrees of severity. NOP is usually described as being located in and around the eye but can progress to the surrounding areas of the face and head. Many comorbid conditions may predispose the patient to NOP, including peripheral neuropathic pain, fibromyalgia, and Sjogren's Syndrome. A need exists for improved treatment of ocular pain, including reducing or eliminating nociceptive ocular pain and NOP.

### SUMMARY

This disclosure provides safer and effective methods and formulations for treating ocular pain in a subject, including nociceptive ocular pain and NOP. This disclosure provides methods and formulations for use in reducing or eliminating ocular pain in a subject, including nociceptive ocular pain and NOP. In various aspects, use of a formulation for treating nociceptive ocular pain and/or NOP in a subject is disclosed. The treatment method includes a step of topically administering an effective amount of a formulation including a selective Nav1.8 inhibitor to an ocular surface of a subject suffering from nociceptive ocular pain and/or NOP. In certain aspects, the Nav1.8 inhibitor is a peripheral nerve desensitizing agent. In certain aspects, the Nav1.8 inhibitor comprises ambroxol and/or a derivative of ambroxol.

In certain aspects, administration of the formulation provides partial preservation of sensation in the eye, including corneal sensation.

In certain aspects, a use or method further includes administering an analgesic.

In various aspects, a formulation including ambroxol and/or a derivative of ambroxol and a cyclodextrin for use in treating nociceptive ocular pain and/or NOP in a subject is disclosed.

In embodiments, the formulation may include ambroxol and/or a derivative of ambroxol in a weight percent of between 0.01% and 10%, based on total weight or total volume of the formulation.

These and other features, aspects, and advantages described herein will become better understood by persons of ordinary skill in the art upon consideration of the following drawings, detailed description, and appended claims.

### BRIEF DESCRIPTION OF THE FIGURES

The drawings accompanying and forming part of this specification are included to depict certain aspects of the invention. A clearer impression of the invention, and of the components and operation of systems provided with the invention, will become more readily apparent by referring to the exemplary, and therefore non-limiting, embodiments illustrated in the drawings. Any features illustrated in the drawings are not necessarily drawn to scale.

FIG. 1 is a graph illustrating the change in rat corneal sensation after topical administration of ambroxol or an anesthetic.

FIG. 2 is a graph illustrating an assessment of postoperative ocular pain after topical administration of ambroxol via a rat eye-wipe assay.

### DETAILED DESCRIPTION

### Overview

Various aspects of the present disclosure relate to methods, formulations, and compositions (e.g., compositions that include the formulation) and use of the formulations and compositions for reducing or eliminating ocular pain in a subject, particularly of the peripheral nerves, including neuropathic ocular pain (NOP). The disclosed formulations and related uses and methods differ from previous treatment approaches for ocular pain. Specifically, the formulations disclosed herein include compounds capable of suppression of repetitive firing and overexcitability of neuronal cells at the ocular surface, which studies indicate is mostly responsible for signaling inflammatory and neuropathic pain. The formulations administered to treat ocular pain provide partial preservation of sensation in the eye, such as corneal sensation, preventing the subject from unknowingly poking, scratching, or overtly rubbing the eye. In one or more aspects, the methods and formulations include the use of an agent which can selectively inhibit voltage-gated sodium nociceptors (Nav)1.8 specifically, and in addition, serve to desensitize peripheral pain nerve excitability, such as ambroxol (trans-4-(2-Amino-3,5-dibrombenzylamino)-cyclohexanol), or a chemical derivative of ambroxol, for example a salt form of ambroxol.

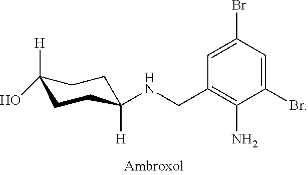

Ambroxol was originally identified as a mucolytic agent and is often used in the treatment of respiratory diseases associated with viscid or excessive mucus. However, there is no prior teaching that ambroxol or its derivatives can be effective when administered topically to treat ocular pain, including NOP. The pharmacological characteristics of ambroxol or its derivatives have never been disclosed previously in relationship to safety of topical application to the ocular surface.

As used herein, the term “analgesic” refers to a compound having an analgesic effect and is well-established for the treatment of pain. Compounds having analgesic activity and that are well-established for the treatment of pain include but are not limited to opioids (e.g., fentanyl, morphine), and non-opioid analgesics such as amide-based local anesthetics (e.g., lidocaine and bupivacaine), ester-based local anesthetics (e.g., tetracaine, proparacaine, and chloroprocaine), and NSAIDS. Ambroxol, while not referred to or considered as an analgesic, has a selective analgesic effect as demonstrated in the current disclosure.

As used herein, the term “local anesthetic” refers to a compound that when administered to a targeted local area of the body induces up to total insensitivity to pain (e.g., or total inhibition of pain) while not affecting sensation beyond the targeted local area. A local anesthetic may induce near total or up to total loss of sensation in the targeted local area. Local anesthetics are a subset of analgesics and include the amide-based anesthetics and the ester-based anesthetics.

The term “tetrodoxin-resistant nociceptors” refers to a type of nociceptor (e.g., a pain receptor) that is not effectively inhibited or blocked by the compound tetrodoxin. An example of a tetrodoxin-resistant nociceptors is the Nav1.8 nociceptor. The term “tetrodoxin-sensitive nociceptor” refers to a type of nociceptor that is effectively blocked by the compound tetrodoxin. An example of a “tetrodoxin-sensitive nociceptor” is the Nav1.7 nociceptor.

The term “selective Nav1.8 inhibitor” refers to a compound having selective inhibition for the Nav1.8 nociceptor over other sensor receptors. For example, ambroxol is selective in blocking the Nav1.8 nociceptor, a tetrodoxin-resistant nociceptor, over tetrodoxin-sensitive nociceptors. Thus, ambroxol is a selective Nav1.8 inhibitor. Opioids, amide-based local anesthetics, ester-based local anesthetics, and NSAIDS are not selective for the Nav1.8 nociceptor over tetrodoxin-sensitive nociceptors, and therefore are not considered to be a selective Nav1.8 inhibitor.

The term “peripheral desensitizing” or “peripheral desensitization” refers to the retraining of a peripheral nerve, such as a peripheral nerve in the eye, so that the nerve is less sensitive to stimuli and/or the dysfunctional, heightened voluntary neuronal activity associated with NOP is suppressed. A peripheral nerve desensitizing agent is a compound that can inhibit an over-sensitized peripheral nerve, effectively desensitizing the peripheral nerve from the over-sensitized state.

Voltage-gated sodium nociceptors (Nav) are important determinants of sensory neuron excitability: they are essential for the initial transduction of sensory stimuli, the electrogenesis of the action potential, and neurotransmitter release from sensory neuron terminals. These channels play a critical role in the development and maintenance of pain. Commonly used topical ophthalmic local anesthetic drugs such as lidocaine, proparacaine and tetracaine are effective due to their nonselective Nav inhibition.

As used herein, the terms “ambroxol derivatives,” “derivatives of ambroxol,” and “chemical derivatives of ambroxol” refer to compounds derived from ambroxol, precursor compounds for ambroxol, and salt forms of ambroxol. For example, chemical derivative of ambroxol may include the acid addition salt ambroxol hydrochloride [trans-4-(2-amino-3,5-dibromobenzyl amino)cyclohexanol hydrochloride] or an N-acylated derivative of ambroxol hydrochloride. In certain aspects, more than one form of ambroxol may be included in a single formulation. For example, in some embodiments, a formulation may include at least a first ambroxol derivative and at least a second ambroxol derivative.

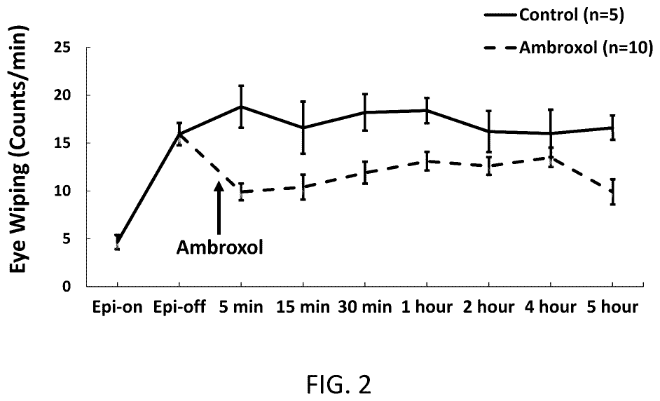

The disclosed methods and formulations are demonstrated through animal studies to be selective in the treatment of ocular pain components, and the methods and formulations do not fully inhibit sensation (e.g., tactile or touch sensation), for example at the ocular surface. The ability of the method and formulation to selectively inhibit pain, including at the ocular surface is advantageous to the subject, offering pain relief to the subject, while still allowing the subject to sense changes in the environment and at the ocular surface (e.g., allowing sensation while relieving pain). Because of this, the methods and formulations may reduce the risk of damaging the eye by allowing the subject to have some sensation of the eye, preventing the subject from unknowingly poking, scratching, or overtly rubbing the eye. In addition, preserving, at least partially, the sensor functions in the eye to changes in temperature, pH and osmolarity (as shown in FIG. 2) is also critical for protecting the eye. For example, hyperosmolarity and increased rate of cooling at the corneal surface that occurs with evaporative thinning of the overlying tear film drive the nociceptive activity that sustains basal tear secretion, which is essential to the ocular surface tear homeostasis and the health of the eye.

“Topical administration”, “topical treatment”, or treatment by a drug for use topically (e.g., a topical anesthetic), as used herein, may refer the administration of a drug or formulation to an exterior surface of a subject (e.g., cornea, skin, or epidermis). “Local administration”, “local treatment”, or treatment by a drug for use locally (e.g., a local anesthetic), as used herein, may refer to topical administration, but also may refer to injection or other introduction into the body at a site of treatment, where the administrated drug has a local effect.

In certain aspects, the formulation is administered in association with surgery or trauma to the eye. In embodiments, an analgesic (e.g., the local anesthetic lidocaine) may also be used topically or systemically. Analgesic compounds, particularly local anesthetics, affect multiple neuronal receptor types and are highly efficacious in removing most, if not all, physical sensations, including tactile (touch), pain, and pressure sensation. Topical anesthetic drugs include but are not limited to the heavily numbing tetracaine, proparacaine, chloroprocaine, lidocaine, and bupivacaine. The formulation may be used alongside the use of local anesthetic or analgesic drugs, locally or systemically, to continue pain relief and to prevent post-surgical injury. For example, in an eye surgery where a local anesthetic drug (e.g., lidocaine) is the primary drug used during surgery, the formulation may be administered near the end of surgery so that when the heavily numbing lidocaine wears off, the formulation continues to provide pain relief while permitting the subject to feel the sensation of the eye, reducing the risk of poking, or scratching the eye by the patient, desiccation (drying up) of the ocular surface, or other external sources of potential injury.

As used herein, the terms ocular, ophthalmic, and optic are used interchangeably to define “of, or about, or related to the eye.” The term ocular pain is an inclusive term encompassing acute and chronic conditions, nociceptive or neuropathic pain of the eye, inclusive of the neural connection between the eye and the brain, involving cellular inflammation, damage, degeneration, or loss.

NOP is not to be confused as a mere description of discomfort symptoms of many disease processes. Rather, it is initiated and/or sustained by dysfunctional elements in the nociceptive system. It is itself a distinct disease entity as opposed to a normal somatosensory “nociceptive pain”. The underlying pathophysiology of neuropathic pain may be a peripheral or centralized sensitization of the nervous system. NOP is frequently seen in illnesses associated with ocular surface tissue damage such as infections, trauma, various ocular surgeries including laser refractive procedures, and chronic severe ocular surface destructions such as limbal stem cell deficiency and sequela of chemical burn. Peripheral injuries trigger complex changes in the central nociceptive system which can lead to centralized sensitization that enhances the sensitivity and responsiveness of the brain regions involved in sensory processing. In some cases, these physiological responses progress to centralized neuropathic pain

Non-ocular conditions have been associated with NOP as well, which include diabetes mellitus and small-fiber peripheral neuropathies, trigeminal neuralgia, and inflammatory auto-immune conditions like Sjogren syndrome and systemic lupus erythematosus. Nociceptive pain, a physiologically normal and protective pain, and neuropathic pain, a physiologically abnormal pain, are not mutually exclusive and can co-exist. Chronic pain usually has a neuropathic component. Tear film abnormalities, anatomic issues, and/or ocular surface inflammation, may lead to the development of changes in corneal nerve structure and function with resulting NOP. However, it should be emphasized that NOP itself is a distinct disease entity. Symptoms of NOP can range from devastating, unrelenting eye pain and severe sensitivity to light (e.g., photophobia) in the worst cases, to mild hyperalgesia (e.g., increased sensitivity to feeling pain and an extreme response to pain) or dysesthesia (e.g., distorted sense, especially to touch). NOP patients may have little or no signs of ocular surface tissue damage, and frequently respond poorly to conventional treatments. Patients suffering from chronic neuropathic pain are characterized by higher health care utilization, higher risk of comorbidities such as depression, anxiety, and sleep disturbances, and lower quality of life compared to patients with chronic non-neuropathic pain.

Although the exact mechanisms of NOP generation are not fully elucidated, input from hyperexcitable peripheral neurons, specifically Nav1.8, is crucial for development, modulation, and maintenance of NOP. Pathological conditions (i.e., nerve injury, inflammation) result in overactivation and disturbed functioning of neuronal and non-neuronal cells, finally contributing to neuronal hyperexcitability, peripheral sensitization, and pain. Hence, dampening overactivity of neurons in the periphery helps reduce the risk factors leading to the development of NOP.

Neuropathic pain, including NOP, is typically not treated with analgesics such as topical or systemic anesthetics (opioid or non-opioids). In fact, topical anesthetics can be used to distinguish nociceptive ocular pain from NOP, especially in the case of centralized sensitization. The presence or worsening of persistent ocular surface pain after a drop of topical anesthetic is placed on the ocular surface suggests a central component to the pain. Incomplete improvement in symptoms after topical anesthetic challenge may indicate a mixed peripheral and central etiology. Although there is an overlap in treatment modalities for nociceptive and NOP, treatment for NOP aims at desensitization of the peripheral and/or central nervous system function. This treatment principle is further described in Harrison Dermer, et al. A Review of Management Strategies for Nociceptive and Neuropathic Ocular Surface Pain. Drugs (2020) 80:547-571; “Addressing the Pain of Corneal Neuropathy”. American Academy of Ophthalmology. July 2010.

Because nerve damage and inflammation can originate in the ocular surface, conventional dry eye treatments including artificial tears and autologous serum eye drops have been employed in attempts to specifically reduce peripheral stimuli (not to target pain directly). Other treatment for peripheral desensitization includes bandage contact lenses and/or non-fenestrated scleral lenses which can insulate the corneal surface from unwanted stimuli. To inhibit or modulate centralized sensitization, systemic use of gabapentin and tricyclic antidepressants have been used to blunt sensory nerve stimulation or the perception of nerve stimulation. Other adjuvant therapies include transcutaneous electrical nerve stimulation, periocular Botulinum toxin, periocular nerve blocks, trigeminal nerve stimulation, intrathecal pain pump, and even cognitive behavioral therapy.

Three types of nociceptor (pain) fibers are found within the cornea including mechanoreceptors (˜20% of total sensory population), polymodal receptors (˜70% of total sensory population), and cold receptors (˜10% of total sensory population). Among all members of Nav family, Nav1.8 has been specifically implicated in neuropathic and inflammatory pain. Nav1.8 is preferentially expressed in polymodal fibers and hence, is expected to be present in about 70% of corneal nociceptors. Since Nav1.8 contributes to repetitive firing and neuronal excitability related to pain, inhibition of Nav1.8 has been shown to control pain transmission and modulate pain perception in the body, likely through the effect on hyperexcitable neurons.

Ambroxol selectively inhibits Nav1.8 over other calcium and sodium channels and has a particularly higher affinity for Nav1.8. For example, amboxol blocks Nav1.8 at approximately 40 times more potently than lidocaine. Although not previously utilized to specifically treat or inhibit ocular pain, the selectivity of ambroxol for Nav1.8 nociceptor, and evidence herein showing the ability of ambroxol formulations to inhibit pain while not eliminating all corneal sensation makes ambroxol-containing compositions an ideal formulation for the treatment, inhibition, and/or amelioration of ocular pain, including NOP.

As used herein, the terms “treat” and “prevent” are not intended to be absolute terms. In embodiments, treatment can refer to a 10%, 20%, 30%, 40%, 50%, 60%, 70%, 80%, 90%, or 100% reduction in the severity of an established disease, condition, or symptom of the disease or condition. In embodiments, a method for treating a disease is considered to be a treatment if there is a 10% reduction in one or more symptoms of the disease in a subject as compared to a control. Thus, the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. In embodiments, references to decreasing, reducing, or inhibiting include a change of 10%, 20%, 30%, 40%, 50%, 60%, 70%, 80%, 90% or greater as compared to a control level and such terms can include but do not necessarily include complete elimination. Treatment can refer to any delay in onset, amelioration of symptoms, improvement in a patient. The effect of treatment can be compared to an individual or pool of individuals not receiving the treatment, or to the same patient prior to treatment or at a different time during treatment.

In one or more aspects, the administration of an effective amount of the formulation results in a decrease in subjective symptom relief (e.g., reduction of ocular pain) or the increase of quality-of-life measures. Quality-of-life measures may include measures determined by the subject completing a quality-of-life survey. Examples of quality-life-surveys include, but are not limited to, the SF-36 survey (Rand Corporation), PROMIS™, Neuro-QoL™, ASCQ-Me™, and NIH Toolbox™, Brief Pain Inventory (BPI), and Brief Ocular Discomfort (BODI) questionnaire. Ocular pain can be quantified using several validated questionnaires including, but are not limited to, visual analog scale (VAS) pain assessment, the Ocular Surface Disease Index© (OSDI©), Neuropathic Pain Symptom Inventory modified for the Eye (NPSI-Eye), the Ocular Pain Assessment Survey (OPAS), and Defense and Veterans Pain Rating Scale (DVPRS), etc.

### Formulations

As discussed above, and in various aspects, compositions and formulations are disclosed for use in the treatment, inhibition, amelioration, and/or prevention of ocular pain including nociceptive ocular pain and NOP in a subject. In embodiments, agent with high selectivity for Nav1.8 inhibition and with potential for peripheral nerve desensitizing can be formulated, such as a drug for treating, ameliorating, inhibiting, or preventing ocular pain. In some embodiments, the formulation is prepared as eye drops.

NOP can be defined as a condition characterized by positive or negative sensory signs, a pain perceived disproportionate to a noxious stimulus, and/or not responsive to analgesics. NOP is a well-known complaint after any corneal surgery. This is mainly due to abnormal regeneration of damaged corneal nerve endings and abnormal connections with adjacent nerve endings which produce spontaneous activity. Tear hyperosmolarity and the resultant ocular surface inflammation can also trigger voluntary activity of corneal nerve endings. NOP may be present in the entire eye, or in a portion of the eye, including but not limited to the cornea and conjunctiva. NOP may arise in the absence of stimuli or in the presence of a typically non-painful stimuli of light (e.g., photoallodynia), burning sensation (e.g., from contact with typically mild chemicals), dryness, or grittiness.

In various aspects, the subject may be any type of animal prone to or suffering from the indicated disease or disorder including but are not limited to mammals, reptiles, amphibians, and birds. For example, the subject may be any type of mammal including but are not limited to humans, non-human primates, cats, dogs, rats, mice, and livestock such as cattle, sheep, goats, and pigs.

In various aspects, the formulation may include a selective Nav1.8 inhibitor and peripheral nerve desensitizing agent (e.g., the Nav1.8 inhibitor acting as a peripheral nerve desensitizing agent). For example, the compositions, formulations, and methods may include a Nav1.8 selective agent, including but are not limited to ambroxol. The use of ambroxol may include any form or salt of ambroxol, including but are not limited to ambroxol hydrochloride or the free acid of ambroxol. The

In various aspects, the concentration of selective Nav1.8 inhibitor and peripheral nerve desensitizing agent, such as ambroxol or ambroxol salt, within the formulation may be between 0.001% and 20%, between 0.01% and 10%, between 0.05% and 5.0%, between 0.1% and 2.5%, between 0.2% and 1%, and between 0.3% and 0.7% by weight based on the total weight or volume of the formulation (w/w, w/v). For example, the concentration of the total concentration of the agent, such as ambroxol or ambroxol salt, may be approximately 0.5% by weight based on the total weight of the formulation (w/w). In another example, the concentration of the total concentration of the agent, such as ambroxol or ambroxol salt, may be between 0.001% and 3.0% by weight based on the total volume of the formulation (w/v).

In various aspects the formulation includes a buffer. The buffer may include any type of buffer system, including but are not limited to borate, citrate, Tris-HCL, phosphate, bicarbonate, lactate, and sodium buffers. The buffers may include any pharmaceutically acceptable salt, acid, or base that is compatible with administration to the ocular surface including but are not limited to boric acid, sodium borate, sodium citrate, potassium phosphate, potassium chloride, and sodium chloride. For example, the buffer may comprise a borate buffer that includes boric acid and sodium borate.

“Pharmaceutically acceptable salts” means salts of compounds which are pharmaceutically acceptable, as defined above, and which possess the desired pharmacological activity. Such salts include acid addition salts formed with inorganic acids, or with organic acids. Pharmaceutically acceptable salts also include base addition salts which may be formed when acidic protons present are capable of reacting with inorganic or organic bases. “Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

In various aspects, the formulation includes cyclodextrins. Cyclodextrins are cylindrical oligosaccharides with a lipophilic central cavity and hydrophilic outer surface. The cyclodextrins can form complexes with lipophilic drugs, sequestering the drugs within the lipophilic cavity, which can enhance solubility and absorption qualities of the formulation. The formulation may include any cyclodextrin type (e.g., α, β, γ, or hydroxypropyl gamma cyclodextrin) and any size of cyclodextrin. For example, the cyclodextrin may have a molecular weight of between 800 and 2400 g/mol, between 1000 and 2000 g/mol, between 1200 and 1800 g/mol, or between 1200 and 1600 g/mol. The formulation may also include other polymers and polymer-types including but not limited to polyethylene glycols (e.g., polyethylene glycol with a molecular weight of approximately 300 g/mol), polysaccharide dextran (e.g., Dextran T500™ sold by Pharmacosmos, having an average molecular weight of approximately 500,000 g/mol), povidone polyvinylpyrrolidone (PVP) (e.g., PVP K30), and cellulose ethers such as carboxymethylcellulose (CMC) Methocel™ and Methocel E4M™, sold by DuPont.

In various aspects, the formulation is prepared to have a specific osmolarity or an osmolarity within a specific range. For example, the formulation may be prepared to have an osmolarity in the range of 270 to 310, 270 to 320, or 280 to 320 mOsm/liter. For instance, the formulation may have an osmolarity of approximately 280, 290, 300, 310, or 320 mOsm/liter.

In various aspects, the formulation may be prepared to have a specific pH or a pH within a specific range. For example, the formulation may be prepared to have a pH in the range of 6.5 to 7.4, 6.5 to 7.0, or 6.8 to 7.4. For instance, the formulation may have a pH of approximately 6.5, 6.6, 6.7, 6.8, 6.9, 7.0, 7.1, 7.2, 7.3, 7.4, or 7.5.

In various aspects, the formulation may be prepared into any type or modality for treatment of the eye. For example, the formulation may be included in an eye drop preparation or an ointment (e.g., an ophthalmic ointment). In another example, the formulation may be impregnated with dissolvable biopolymers for the slow release onto the tear film as an ocular insert that is applied topically to the eye (e.g., topical administration). For instance, the biopolymer may include hydrogel.

Topical administration refers to administration by application of the formulation to the epidermis (e.g., the eyelids) or to the ocular surface (e.g., the cornea and conjunctiva). These types of compositions are typically in the form of solutions, gels, drops, creams, ointments, suspensions, dispersions, nanoparticulate dispersions, emulsions, microemulsions, liposomes, pastes, and sprays. Topical administration of drug for effecting ocular pain in clinical practice has undoubted advantages, including reduction in or absence of systemic side effects due to low or no systemic absorption, relatively few drug-drug interactions, ease of use and dose determination, avoidance of first-pass metabolism, improved patients' compliance, and direct access to the target site.

Frequency of topical application to a subject (for example, a human or animal) may vary between patients. For example, in some circumstances, the disclosed formulations may be applied as frequently as once every ten minutes or as infrequently as once every day. The formulation may be applied once, twice, three times or more over a period of one day, two days, three days, four days, five days, or more than five days. The disclosed formulations may also, in some embodiments, include an extended-release vehicle, such as a biocompatible polymer, dissolved in the carrier or by itself impregnated with ambroxol to hold the ambroxol and slowly release it into the tear film or onto the ocular surface, preferably for an extended-release period of up to six months or longer. The biocompatible polymer may be biodegradable or non-biodegradable, depending on desired use and application schedule. Example biocompatible polymers that may be used in the disclosed formulations as an extended-release vehicle include but are not limited to poly-2-hydroxyethylmethacrylate (p-HEMA hydrogels), poly(lactic-co-glycolic) acid (PLGA), polycaprolactone (PCL), hydroxypropyl cellulose, anecortave acetate (AnA), gelatin, and/or collagen. The inclusion of an extended-release vehicle may, in some cases, allow for less frequent application while still providing therapeutic efficacy for ocular pain including NOP. For instance, the biopolymer may include hydrogel. Other preparations, formulations, and components of formulation may include those listed in United States Patent No. U.S. Pat. No. 10,265,280, which is incorporated by reference in its entirety.

In one or more aspects, the disclosed formulations may also include one or more additives. Additives that may be included in the disclosed formulations include but are not limited to demulcents, preservatives, emollients, ionic species, pH-adjusting agents, and other possible additives. Example demulcents that may be used in the disclosed formulations include but are not limited to glycerin, carboxymethylcellulose, hydroxyethyl cellulose, hydroxypropyl methylcellulose, methylcellulose, dextran 70, gelatin, polyethylene glycol 300, polyethylene glycol 400, polysorbate 80, polyvinyl alcohol, povidone, cyclodextrins, etc. Examples of preservatives that may be used in the disclosed formulations include benzalkonium chloride and chlorobutanol. Emollients may include lanolin preparations, various mineral oils, Omega 3, paraffin, petroleum and waxes. Zinc and or sodium ions may be included in the disclosed formulation as an ionic species, in some embodiments. Examples of pH-adjusting agents that may be used include citrate buffers (e.g., sodium citrate), borate buffers (e.g., sodium borate), and other acidic or basic compounds. The formulation may include one or more chelating agents including but not limited to EDTA (e.g., edetate disodium) and EGTA (e.g., ethylene glycol tetraacetic acid). The formulation may include one or more surfactants including but not limited to sodium dodecyl sulfate. Numerous configurations and variations of additives may be used in the disclosed formulations.

Another aspect of the present invention is directed to a pharmaceutical composition that includes a therapeutically effective amount of ambroxol or a pharmaceutically acceptable amount of ambroxol and a pharmaceutically acceptable carrier. The term “therapeutically effective amount” refers to an amount of a compound that is effective in producing the desired therapeutic response in a particular patient suffering from a disease or disorder (e.g., eye pain including NOP).

The term “therapeutically effective amount” or “effective amount” includes the amount of the compound of the present disclosure or a pharmaceutically acceptable salt or a stereoisomer thereof, that when administered, induces a positive modification in the disease or disorder to be treated (e.g., reduction of eye pain), or is sufficient to prevent development or progression of the disease or disorder, or alleviate to some extent, one or more of the symptoms of the disease or disorder being treated in a subject. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control. If, after a period of time, the effective amount of compound no longer demonstrates the decrease in the symptoms of the disorder or condition (e.g., eye pain) as stated above, then the compound is considered to be no longer effective. For example, the formulation may still continue to treat ocular pain in a subject after the co-administered analgesic is no longer effective.

The terms “pharmaceutically acceptable carrier”, “carrier”, or medium, as known in the art, refer to any pharmaceutically acceptable material, medium, composition, or vehicle, suitable for administering to a subject. Suitable carriers may include, for example, liquids (both aqueous and non-aqueous alike, and combinations thereof), solids, encapsulating materials, gases, and combinations thereof (e.g., semi-solids), and gases, that function to carry or transport the compound from one organ, or portion of the body, to another organ, or portion of the body. The terms may also refer to reagents, buffers, cells, compounds, materials, compositions, and/or dosage forms that are not only compatible with other agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio.

In various aspects, the formulation may include a pharmaceutically acceptable excipient. The term “pharmaceutically acceptable excipient” refers to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, salt solutions (such as Ringer's solution), alcohols, oils, gelatins, carbohydrates, fatty acid esters, hydroxymethycellulose, polyvinyl pyrrolidine, and colors. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances. One of skill in the art will recognize that other pharmaceutical excipients are useful in the present invention.

### Methods

In various aspects, as disclosed herein, the formulation is administered for the treatment of ocular pain including NOP, employing one or more of the formulation or formulation components described above. In certain aspects, the formulation is topically administered to the eye and eyelids of a subject, the subject including any type of animal (e.g., human) as detailed above. In various aspects, the formulation includes a selective Nav1.8 inhibitor and peripheral nerve desensitizing agent. For example, the Nav1.8 inhibitor compound ambroxol and/or a salt form of ambroxol acts as a peripheral nerve desensitizing agent in the treatment of NOP. The concentration of ambroxol and/or the salt form of ambroxol in the formulation may be within the range or approximation of any concentration as described in detail above. The formulation may also include any component, buffer, carrier, cyclodextrin, polymer, surfactant, preservative, or salt as described in detail above. One embodiment is a method for treating ocular pain, in a subject by topically administering a formulation described above that includes a selective Nav1.8 inhibitor and peripheral nerve desensitizing agent (e.g., the selective Nav1.8 inhibitor acting as the peripheral nerve desensitizing agent) to an ocular surface of a subject suffering from ocular pain. In embodiments, the selective Nav1.8 inhibitor and peripheral nerve desensitizing agent comprises ambroxol and/or a salt form of ambroxol.

In one or more aspects, the administration of the formulation is associated with an ocular surgery. The ocular surgery may include any type of surgical intervention involving the eye including but are not limited to LASIK (Laser-Assisted In Situ Keratomileusis), PRK (Photorefractive Keratectomy), LASEK (Laser-Assisted Subepithelial Keratectomy), Epi-LASIK (Epithelial Laser In Situ Keratomileusis), wavefront-guided LASIK, wavefront-guided PRK, PTK (Phototherapeutic Keratectomy), Intracorneal ring segments (ICRS) implantation, corneal cross-linking, conductive keratoplasty (CK), INTACS (Intrastromal corneal ring segments) implantation, corneal tattooing, keratectomy, keratoplasty, keratoprosthesis (artificial cornea) implantation, pterygium excision, chalazion excision, dacryocystorhinostomy (DCR), canalicular laceration repair, punctal plug insertion, removal of a foreign body from the eye, and surgeries for cataract, glaucoma and retinal diseases. For example, the formulation may be used to treat, prevent, inhibit, or ameliorate ocular pain including NOP associated with eye surgery.

In one or more aspects, the administration of the formulation is associated with ocular trauma or sequelae involving severe destruction of the ocular surface or globe wall, such as corneal abrasion or erosion, penetration, perforation, foreign bodies, ruptured globe, chemical burns (e.g., alkaline or acid spills into the eye) or heat injury of the ocular surface. In one or more aspects, the administration of the formulation is associated with treating ocular infections (e.g., bacterial, fungal, viral, and parasitic), such as in infectious keratitis. For example, the formulation may be administered for subjects with infections (e.g., from herpes simplex, zoster viruses, or acanthamoeba) that can lead to recalcitrant ocular pain and are especially prone to develop NOP. In one or more aspects, the administration of the formulation is associated with conditions associated with or leading to acute or chronic inflammations, such as recurrent corneal erosions/epithelium-basement membrane dystrophies, ocular surface diseases (e.g., Sjogren syndrome, autoimmune/connective tissue diseases, Steven-Johnson syndrome, pemphigoid), limbal stem cell deficiency and uveitis of all etiologies.

In some aspects, the methods and use of the formulation or composition further include the administration or co-administration of an analgesic such as opioids (e.g., fentanyl and morphine), or non-opioid analgesics such as amide-based local anesthetics (e.g., lidocaine and bupivacaine), ester-based local anesthetics (e.g., tetracaine, proparacaine, and chloroprocaine), or pain-relieving NSAIDS). For example, the analgesic may be administered during a treatment of an eye such as eye surgery, or after eye trauma or eye surgery, where the formulation has also been administered. For example, the formulation may be topically administered to the ocular surface before, approximately the same time as, or after the analgesic is administered. The formulation may also be topically administered before the onset of postoperative pain or eye trauma pain, wherein the onset of postoperative pain or eye trauma pain has been delayed by the administration of the analgesic. For example, after eye surgery where a local anesthetic is used, ambroxol is administered so that when the local anesthetic wears off, the ambroxol is still effective in relieving pain while retaining some sensation of the eye. In another example, after eye trauma, where a local anesthetic may be initially used to reduce intense eye pain (e.g., while the patient is being overseen by personnel to watch for the subject reaching to touch or scratch the eye), ambroxol is administered so that when the local anesthetic wears off, the ambroxol is still effective in relieving pain while retaining some sensation of the eye.

When administered along with the formulation, analgesics may be administered either locally or systemically. For example, the subject may receive an opioid (e.g., fentanyl) intravenously while receiving a topical application of the formulation to the eye. In another example, the subject may receive a non-opioid local anesthetic (e.g., intramuscular lidocaine) while receiving a topical application of the formulation to the eye. In another example, the subject may receive a local anesthetic in the form of proparacaine-containing eye drops while also receiving a topical application of the formulation to the eye.

By administering the formulation at a point before other analgesics wear off (e.g., the formulation extending pain relief after the analgesic administered before wears off), the formulation continues to provide pain relief (e.g., NOP relief), while still allowing the subject to experience certain key sensations of the surface of the eye, such as sensation to cooling and hyperosmolarity. The analgesic administered before the administration of the formulation may include any opioid and non-opioid analgesic for eye pain including but are not limited to tetracaine, proparacaine, lidocaine, or bupivacaine. The formulation may include or exclude other analgesics.

In one or more aspects, the formulation is administered for eye trauma or other eye pain not related to surgery. For example, the formulation may be used for eye trauma caused by physical stress (e.g., corneal abrasion, chemical, and burns). The formulation may also be administered to subjects for treating NOP related to trauma and/or unexplained NOP.

In one or more aspects, the formulation is used in conjunction with other classes of analgesic (e.g., non-opioids or opioids) after an eye trauma event (e.g., cornea abrasion, eye bruise, eye puncture, eye scratch). For example, the formulation may be used before, during, or after the administration of other classes of analgesics. For instance, after an eye trauma event, the formulation may be used after an analgesic (e.g., lidocaine or a NSAID) is administered.

In one or more aspects, the formulation may be administered within six hours, within four hours, three hours, within two hours, within one hour, within 45 minutes, within 30 minutes, within 15 minutes, within 10 minutes, within 5 minutes, within two minutes, within one minute, within 30 seconds, or within 10 seconds of the subject receiving eye trauma or eye surgery, or receiving other classes of analgesics, topically or systemically. For example, the subject may first receive an analgesic (e.g., lidocaine) after an eye puncture, then receive an ambroxol-containing formulation an hour after the administration of the analgesic (as the analgesic begins to wear off).

In another example, where an extended-release vehicle is included in the formulation, the formulation may be topically applied with a frequency of at least one time a day to one time a week, or one time a month to one time every six months. Frequency of topical application can be determined by numerous considerations, including level of symptom relief provided, pain relief experienced, as well as other pertinent health considerations, such as possible drug interactions.

In one or more aspects, a formulation is administered using any desired technique. For example, the formulations may be eye drops that are administered manually by a user. In other cases, the formulations may be topically applied as a gel or ointment directly to a desired region of or around the eye, for example using a swab or other type of applicator. In some aspects, the disclosed formulations may be delivered using a device designed for immediate formulation release or extended formulation release. For example, the formulation may be delivered using one or more of the following devices: external pumps, contact lenses, punctal plugs, muco-adhesive tablets, pills, capsules, pellets, particles, plasters, an ocular insert device, strips placed onto the lids, conjunctiva or cornea, conjunctival inserts or depots, subconjunctival, subtenon, and intravitreal injections, or another suitable types of devices. For instance, the device may be configured to administer the formulation to a subject.

In one or more aspects, a percentage of ocular sensation, including corneal sensation, is preserved after the formulation is administered (e.g., the formulation provides partial preservation of corneal sensation in the eye). For example, upon the administration of the formulation to the subject's eye, some corneal sensation is preserved in the subject, even at a time when the effect of the formulation is at the maximal. For instance, if the maximal effect of pain reduction by the formulation is at five minutes after administration of the formulation to the eye, the subject will still feel corneal sensation at five minutes. The ability of the subject to sense the cornea is a safety advantage, as a subject having a total loss of physical sensation, such as when a local anesthetic (e.g., proparacaine) is administered, may result in the subject unknowingly damaging the eye by rubbing, poking, or scratching. Preserving ocular sensation to changes in pH, temperature and osmolarity is also physiologically essential in the maintenance of ocular surface health and vision, as hyperosmolarity and increased rate of cooling at the ocular surface, such as corneal surface, drive the nociceptive activity that sustains basal tear secretion. The maximal ocular sensation retained or preserved after administration of the formulation can be equal to or greater than 1.0, 2.0, 3.0, 4.0, 5.0, 7.5, 10.0, 12.5, 15.0, 17.5, 20.0, 25.0, 30.0, 35.0, 40.0, 45.0, or 50%. The maximal ocular sensation retained or preserved after administration of the formulation can range from 5% to 50%, from 10% to 30%, from 10% to 20%, from 5% to 15%, and from 15% to 20%. For example, a subject having had eye surgery may be administered the formulation that reduces the amount of ocular sensation by 90%, with preservation of ocular sensation (e.g., tactile sensation) in the eye being at least or approximately 10%. In another example, a subject having had eye surgery may be administered the formulation that reduces the amount of corneal sensation by 85%, with preservation of corneal sensation in the eye (e.g., tactile sensation) being at least or approximately 15%. In another example, a subject having had eye surgery may be administered the formulation that reduces the amount of corneal sensation by 80%, with preservation of corneal sensation (e.g., tactile sensation) in the eye being at least or approximately 20%.

In some aspects of the disclosure, the methods, compositions, and formulations may be defined in any of the following numbered paragraphs:

1. Use of a formulation for treating nociceptive and/or neuropathic ocular pain in a subject comprising:
topically administering an effective amount of a formulation comprising a selective Nav1.8 inhibitor to an ocular surface of a subject suffering from nociceptive and/or neuropathic ocular pain, wherein the Nav1.8 inhibitor is a peripheral nerve desensitizing agent, wherein the Nav1.8 inhibitor is ambroxol and/or a derivative of ambroxol.

2. The use of paragraph 1, wherein administration of the formulation provides partial preservation of sensation in the eye.

3. The use of paragraph 2, wherein partial preservation of sensation in the eye comprises a preservation of at least 10% of sensation in the eye.

4. The use of paragraph 2, wherein partial preservation of sensation in the eye comprises a preservation of at least 20% of sensation in the eye.

5. The use of any of paragraphs 1-4, wherein the ambroxol and/or a derivative of ambroxol is in a weight percent of between 0.001% and 10%, based on total weight or total volume of the formulation.

6. The use of any of paragraphs 1-5, wherein the ambroxol and/or a derivative of ambroxol is in a weight percent of between 0.05% and 2.0%, based on total weight or total volume of the formulation.

7. The use of any of paragraphs 1-6, wherein the formulation further comprises a cyclodextrin.

8. The use of any of paragraphs 1-7, wherein an administration of the formulation is associated with eye trauma or eye surgery.

9. The use use of any of paragraphs 1-8, further comprising administering an analgesic.

10. The use of paragraph 9, wherein the formulation is topically administered to the ocular surface before the analgesic is administered.

11. The use of paragraph 9, wherein the formulation is topically administered at approximately the same time that the analgesic is administered.

12. The use of paragraph 9, wherein the formulation is topically administered after the analgesic is administered.

13. The use of paragraphs 9-12, wherein the formulation is topically administered before the onset of postoperative pain or eye trauma pain, wherein the onset of postoperative pain or eye trauma pain has been delayed by the administration of the analgesic.

14. The use of paragraphs 9-13, wherein the formulation continues to treat nociceptive and/or neuropathic ocular pain after the effect of the analgesic no longer effective.

15. The use of paragraphs 9-15, wherein the analgesic comprises tetracaine, proparacaine, chloroprocaine, lidocaine, or bupivacaine.

16. The use of any of paragraphs 9-15, wherein the analgesic comprises an opioid.

17. The use of any of paragraphs 9-15, wherein the analgesic comprises a non-steroidal anti-inflammatory drug.

18. The use of any of paragraphs 1-17, wherein the formulation is administered within 60 minutes of the subject experiencing eye trauma or eye surgery.

19. The use of any of paragraphs 1-17, wherein the formulation is administered within 20 minutes of the subject experiencing eye trauma or eye surgery.

20. The use of any of paragraphs 1-17, wherein the formulation is administered within 5 minutes of the subject experiencing eye trauma or eye surgery.

21. The use of paragraph 1-20, wherein a device provides the application of the formulation.

22. The use of any of paragraphs 1-21, wherein the formulation includes a biocompatible polymer.

23. The use of paragraph 22, wherein the ambroxol and/or a chemical derivative of ambroxol is impregnated within the biocompatible polymer.

24. The use of paragraph 22 or 23, wherein the biocompatible polymer is dissolved in a carrier.

25. The use of any of paragraphs 22 to 24, wherein the biocompatible polymer comprises poly-2-hydroxyethylmethacrylate (p-HEMA hydrogels), poly(lactic-co-glycolic) acid (PLGA). polycaprolactone (PCL), hydroxypropyl cellulose, anecortave acetate (AnA) gelatin, and/or collagen.

26. The use of any of paragraphs 1-25, wherein the formulation further comprises an additive, wherein the additive is a demulcent, preservative, emollient, or pH-adjusting agent.

27. The use of any of paragraphs 1-26, wherein the administration of the effective amount of the formulation results in a decrease in subjective symptoms or the increase of quality-of-life measure.

28. Use of the formulation of paragraph 1 in the manufacture of a medicament for the treatment of nociceptive and/or neuropathic ocular pain in a subject, wherein the treatment comprises topically administering an effective amount of the formulation to an eye of the subject.

29. A formulation for the use in paragraph 1 comprising a composition, the composition comprising:
ambroxol and/or a derivative of ambroxol in a weight percent of between 0.01% and 10%, based on total weight or total volume of the formulation; and a cyclodextrin.

### EXAMPLES

The following examples are illustrative and should not be interpreted to limit the scope of the claimed subject matter.

### Example 1: Formulations of the Selective Nav1.8 Inhibitor and Peripheral Nerve Desensitizing Agent

The pharmaceutical properties of ambroxol and its derivatives are independent of other ingredients included in the formulations. Combinations of various ingredients do not significantly affect the pharmaceutical properties of ambroxol and its derivatives.

Tables 1 and 2 disclose exemplary formulations for the treatment of ocular pain including NOP:

TABLE 1

| Component | % Concentration* |
|---|---|
| Ambroxol HCl | 0.2-0.5% |
| Polyethylene Glycol (PEG) 300MW | 1% |
| Dextran T500 | 0.1% |
| Boric Acid | 1.5% |
| Sodium Borate | 0.05% |
| Sodium Chloride | 0.05% |
| Potassium Chloride | 0.1% |
| Edetate Disodium | 0.05% |
| Benzalkonium Chloride | 0.01% |
| Sterile Water | To Volume |

*Based on total weight or total volume of the formulation; pH of the formulation is adjusted to fall between 6.5-7; osmolarity is in the range between 270 to 320 mOsm/L, in particular at approximately 290 mOsm/L.

TABLE 2

| Steps | Component | % Concentration |
|---|---|---|
| 1 | Potassium Phosphate Monobasic | 0.130-0.260 |
| | Anhydrous, | |
| 2 | Ambroxol HCl | 0.270-0.540 |
| 3 | Potassium Phosphate Dibasic, Trihydrate | 0.280-0.510 |
| 4 | Potassium Chloride | 0.760 |
| 5 | Alginate (Sigma) | 0.500 |
| Or | PVP K30 | 0.2-1.0 |
| Or | CMC | 0.2-1.5 |
| | Purified Water | Q.S. to 100% w/w |

*Based on total weight or total volume of the formulation, pH = 6.8-7.4, Osmolarity = 270-320 mOsm/L.

The concentration of components of the formulation comprising ambroxol may vary. Table 3 details ranges of formulation components for the treatment of ocular pain including nociceptive ocular pain and/or NOP:

TABLE 3

| Component | % Concentration range* |
|---|---|
| Ambroxol HCl | 0.001%-20.0% |
| Polyethylene Glycol (PEG) 300MW | 0.01% 10.0% |
| Dextran T500 | 0.01% to 1.0% |
| Hydroxypropyl Gamma Cyclodextrin | 0.1% to 20% |
| Methocel E4M | 0.15% to 10% |
| Boric Acid | 0.01% to 3.0% |
| Sodium Borate | 0.01% to 5.0% |
| Sodium Chloride | 0.01% to 2.0% |
| Potassium Chloride | 0.01% to 1.0% |
| Edetate Disodium | 0.005% to 1.0% |
| Benzalkonium Chloride | 0.00%-0.10% |
| Sterile Water | To Volume |

*Based on total weight or total volume of the formulation

### Example 2: Partial Preservation of Corneal Sensation with Formulation Containing Ambroxol

Acute ocular pain, defined as pain of a few days in duration, is commonly caused by trauma (including eye surgeries), infection and inflammation. Whereas chronic pain, often longer than 2 weeks, is commonly caused by chronic diseases that incite inflammation and damage to the ocular surface, such as dry eye disease, herpetic keratitis, post-laser refractive surgery. Symptoms include discomfort, burning, pain, itching, and even photophobia. Ocular pain can have drastic impact on a person's quality of life, productivity, and ability to work.

To treat ocular pain, opioid and non-opioid local or topical anesthetics can be used for various ocular procedures to provide temporary relief of pain, but prescription of topical anesthetics (e.g., proparacaine or tetracaine) or NSAIDs are discouraged by physicians due their high rate of adverse effects. Unsupervised use of topical anesthetics or NSAIDs can cause corneal ulcers, infections, even perforation. Oral opioid or non-opioid pain medications also pose serious systemic side effects such as overdose and drug-drug interactions and can sometime lead to dependency and abuse.

Research has shown that ambroxol can stimulate water and mucin secretion across the conjunctiva and has anti-inflammatory properties suitable for treatment of dry eye disease. Additionally, ambroxol is an inhibitor of neuronal Nav nociceptors, preferentially for Nav1.8, which is responsible for sending inflammatory and neuropathic pain signals and hence has analgesic effect. The ability of ambroxol to preserve certain sensor functions of the cornea is specifically promising since it carries a much higher safety profile.

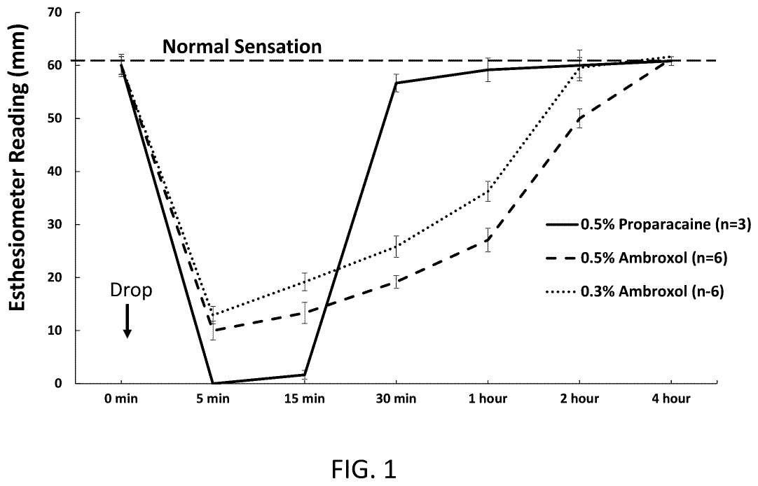

The ability of an ambroxol-containing formulation to partially preserve rat corneal sensation is shown in FIG. 1. The action of ambroxol preserves partial sensations of cornea, presumably due to its selectivity on certain Nav nociceptors, particularly on Nav1.8. In addition, other nociceptors such as transient receptor potential vanilloid subtype 1 (TRPV1) also participate in pain sensing and transmission. No data to date suggests that ambroxol binds to TRPV1 receptors.

The nerve system at the ocular surface is more unique than any other parts of the body in its principal role of protecting, sustaining, and restoring the optical tear layer. To meet this existential need, the cornea has evolved into a most powerful, sensitive, and complex pain system, unparallel in any other parts of the body. Due to this uniqueness of the cornea, utility of drugs administered to the ocular surface is difficult to ascertain without experimentation. The applicability, efficacy and safety of these drugs cannot be predicted or assumed. For example, it is well-known that the efficacy of an analgesic or local anesthetic in other parts of the body cannot be simply replicated at ocular surface and directly applied to the eye to control ocular pain. In the case of a nonselective sodium channel blocker anesthetic, without considering their toxicity to the cornea, if such delicate sensor network in the cornea is completely blocked (e.g., proparacaine in FIG. 1), the ocular surface would lose its ability to maintain adequate basal tear secretion, react properly to external insults and fail to protect the eye. Being able to sense foreign objects (e.g., touch and pain), temperature alteration or change in tear chemical components (e.g., pH and osmolarity) and respond by avoidance and/or increasing tear secretion is extremely critical in the protection of eye and vision. Our discovery of the unexpected pharmacological property of ambroxol in partial preservation of corneal sensation constitutes an important safety feature for topical administration, especially for ocular pain control. This new finding is unique and has not been disclosed previously to the best of our knowledge.

In this Example, 0.3% and 0.5% ambroxol HCl solutions were prepared with PEG 300MW, Dextran T500 and borate buffer as main ingredients. Commercially available 0.5% proparacaine HCl was used as topical anesthetic control. Corneal sensation was assessed by Cochet-Bonnet esthesiometer before and at different time points (e.g., 0 minutes, 5 minutes, 15 minutes, 30 minutes, 1 hour, 2 hours, or 4 hours) after one drop of 0.5% proparacaine, 0.5% or 0.3% ambroxol was administered to the eyes of rats. Cochet-Bonnet esthesiometer measures tactile sensation of the cornea. At each time point, corneal esthesiometry was performed twice on each eye and the average value taken as the final reading. Note a minimum of 15% total corneal sensation was preserved in both ambroxol treatment groups even when the effect of ambroxol-containing formulations is at its maximal (5-15 minutes), and higher levels of sensation preservation maintained throughout the duration (about 4 hours) of drug action (FIG. 1).

### Example 3: Selective Inhibition by Ambroxol-Containing Formulation on Peripheral Neuronal Excitability

With minor modification of established rat NOP models, ocular pain was created by removing corneal epithelium (Epi-off) under general anesthesia. 2M NaCl saline (in contrast to 5M NaCl used in previous studies) was used to trigger peripheral neuronal excitement in response to hyperosmolarity while minimizing the potential inherent irritancy to the cornea from 5M NaCl. Then, one hour was given to each rat to allow complete recovery from anesthesia before proceeding to subsequent study steps. Compared to before epithelium removal (Epi-on), corneal sensitivity to pain (eye wiping counts/min) increased significantly after surgery (Epi-off) in response to 2M NaCl. Then, a single drop of 0.5% ambroxol was applied topically on the ocular surface, eye wiping behavior in response to 2M NaCl was monitored continuously by videorecording at various time points (e.g., 5 minutes, 15 minutes, 30 minutes, 1 hour, 2 hours, 4 hours, or 5 hours) and compared with the control group treated with one drop of carrier buffer solution without ambroxol. After treatment with 0.5% ambroxol, while the sensitivity to hyperosmolarity at the ocular surface was obviously preserved, significant reduction in eye wiping count/min was documented, lasting for at least 4 hours. This indicates that ambroxol can inhibit peripheral pain signal activity and hence dampen pain signal transduction centrally to the brain in response to hyperosmolarity.

Even at the maximal effect of ambroxol-containing formulation (up to 30 min after administration), it was demonstrated that neuronal sensation for hyperosmolarity was preserved at the ocular surface and such signals transmitted centrally to the brain (increased eye wiping behavior triggered compared to Epi-on). This observation revealed that pain signal activity was significantly dampened (comparing to carrier buffer control group), suggesting potential for peripheral nerve desensitization in treating NOP. When using 0.5% proparacaine HCl to conduct the same study, at its maximal effect (5 min) no significant change was seen in eye wiping counts before (Epi-on) and after surgery (Epi-off) (data not shown), indicating nonselective inhibition of neuronal excitability at the ocular surface. Due to its short duration of action, a proparacaine group was not included in this study (FIG. 2).

The ability of an ambroxol-containing formulation in reducing postoperative ocular pain as assessed via an eye-wipe assay is demonstrated in FIG. 2. Here, the formulation is shown to 1) preserve the sensation to elevated osmolarity at the ocular surface; and 2) dampen peripheral neuronal pain signal activity leading to decreased pain signal firing centrally to the brain. This dampening of peripheral neuronal excitability suggests that ambroxol has great potential as a peripheral nerve desensitizating drug, specifically for NOP. These results indicate that ambroxol-containing formulations can inhibit peripheral neurosensory excitability when administered topically on the ocular surface, presumably by preferentially blocking Nav1.8 nociceptor, therefore possessing the potential for becoming a peripheral nerve desensitization medicine for NOP. Nav1.8 nociceptor in the nerve tissue contributes to repetitive firing and neuronal excitability and correlate significantly with neuropathic and inflammatory pain (Bird E V, et al., Mol Pain. 2013; Daou I, et al., eNeuro. 2016).

Ambroxol has the same level of binding affinity for the Nav1.7 nociceptor as lidocaine. However, ambroxol is 40 times more potent in binding the Nav1.8 nociceptor than lidocaine. Although the selective blocker ambroxol appears less potent in treating ocular pain, it is exactly this unique pharmacological property that renders a previously unknown safety advantage of ambroxol for ocular pain control. For the same reason, a nonselective blocker lidocaine may not be suited for peripheral nerve desensitization in treating NOP due its low potency on the dysfunctional, overly excited Nav1.8 nociceptor implicated in NOP. The concentration of lidocaine has to be raised to such a high level to achieve similar effects which as a result may pose unacceptable safety risks to ocular surface tissues.

These results also demonstrate a feature of the ability of the formulation to preserve key physiological sensations, including hyperosmolarity. This ability of the formulation to preserve multiple physiological sensations constitutes a newly discovered safety feature of ambroxol when used topically for ocular pain control, both nociceptive and neuropathic.

The rat eye-wipe assay is an established animal behavioral and neurophysiological tool for the assessment of corneal sensitivity to noxious stimuli (Price T J, et al., Br. J. Pharmacol.). Increased corneal sensitivity was shown in male rats following lacrimal gland excision which correlates with eye wiping behaviors in response to hypertonic saline (5M NaCl) (Meng I D, et al., Invest. Ophthalmol. Vis. Sci. 2015).

Overall, the discovery of the partial preservation of tactile sensation at the cornea (FIG. 1) and the preservation of other key corneal sensations, specifically that to hyperosmolarity (as shown in FIG. 2), constitute unexpected results for ambroxol. The property of ambroxol for partially preserving tactile sensation was previously unknown and provides a newly discovered safety feature for ambroxol. Therefore, this application of ambroxol for ocular pain control in general is uniquely demonstrated.

It is contemplated that any embodied method, formulation, composition, or article described herein can be implemented with respect to any other method, formulation, composition, or article described herein. The methods described herein may include additional steps or may exclude one or more steps, and the steps may be performed in any order.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention.

While the present invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed. To the contrary, it should be appreciated that many equivalents, alternatives, variations, and modifications, aside from those expressly stated, are possible and fall within the spirit and scope of the invention as defined by the appended claims.

### References

All references listed in this application are incorporated in whole by reference for all purposes, as long as they do not conflict with the invention. While specific embodiments and examples of the disclosed subject matter have been discussed herein, these examples are illustrative and not restrictive. Many variations will become apparent to those skilled in the art upon review of this specification and the claims below.
1. “Addressing the Pain of Corneal Neuropathy”. American Academy of Ophthalmology. July 2010. Retrieved 2018 Apr. 16.
2. Rosenthal, Perry; Borsook, David (2016). “Ocular neuropathic pain”. British Journal of Ophthalmology. 100(1):128-134.
3. Kalangara, Jerry P.; Galor, Anat; Levitt, Roy C.; Felix, Elizabeth R.; Alegret, Ramon; Sarantopoulos, Constantine D. (2015). “Burning Eye Syndrome: Do Neuropathic Pain Mechanisms Underlie Chronic Dry Eye?”. Pain Medicine. pp. 746-755.
4. “Neuropathic dry eye: When serum defeats tears”. Ophthalmology Times. Retrieved 2018 May 2.
5. Meng I D, Barton S T, Mecum N E, Kurose M. Corneal sensitivity following lacrimal gland excision in the rat. Invest Ophthalmol Vis Sci. 2015;56:3347-3354.
6. Daou I, Beaudry H, Ase A R, Wieskopf J S, Ribeiro-da-Silva A, Mogil J S, Séguéla P. Optogenetic Silencing of Nav1.8-Positive Afferents Alleviates Inflammatory and Neuropathic Pain. eNeuro. 2016 Mar. 16; 3(1):ENEURO.0140-15.2016.
7. Joshi S K, Honore P, Hernandez G, Schmidt R, Gomtsyan A, Scanio M, Kort M, Jarvis M F. Additive antinociceptive effects of the selective Nav1.8 blocker A-803467 and selective TRPV1 antagonists in rat inflammatory and neuropathic pain models. J Pain. 2009 March; 10(3):306-15.
8. Mecum N E, Russell R, Lee J, Sullivan C, Meng I D. Optogenetic Inhibition of Nav1.8 Expressing Corneal Afferents Reduces Persistent Dry Eye Pain. Invest Ophthalmol Vis Sci. 2021 Nov. 1; 62(14):15.
9. Price T J, Patwardhan A, Akopian A N, Hargreaves K M, Flores C M. Modulation of trigeminal sensory neuron activity by the dual cannabinoid-vanilloid agonists anandamide, N-arachidonoyl-dopamine and arachidonyl-2-chloroethylamide. Br. J. Pharmacol. 2004; 141:1118-1130.
10. Bird E V, Christmas C R, Loescher A R, et al. Correlation of Nav1.8 and Nav1.9 sodium channel expression with neuropathic pain in human subjects with lingual nerve neuromas. Mol Pain. 2013 Oct. 21; 9:52.
11. Dermer H, Lent-Schochet D, Theotoka D, Paba C, Cheema A A, Kim R S, Galor A. A Review of Management Strategies for Nociceptive and Neuropathic Ocular Surface Pain. Drugs. 2020 April; 80(6):547-571. Doi:10.1007/s40265-020-01282-3. PMID:32207101.
12. Rosenthal P, Borsook D. Ocular neuropathic pain. Br J Ophthalmol. 2016 January; 100(1):128-34. doi:10.1136/bjophthalmol-2014-306280. Epub 2015 May 5. PMID:25943558; PMCID:PMC4717373.
13. Ebrahimiadib N, Yousefshahi F, Abdi P, Ghahari M, Modjtahedi B S. Ocular Neuropathic Pain: An Overview Focusing on Ocular Surface Pains. Clin Ophthalmol. 2020 Sep. 25; 14:2843-2854. doi:10.2147/OPTH.S262060. PMID:33061269; PMCID:PMC7524198.
14. Galor A, Levitt R C, Felix E R, Martin E R, Sarantopoulos C D. Neuropathic ocular pain: an important yet underevaluated feature of dry eye. Eye (Lond). 2015 March; 29(3):301-12. Doi:10.1038/eye.2014.263. Epub 2014 Nov. 7. PMID:25376119; PMCID:PMC4366454.
15. Mehra D, Cohen N K, Galor A. Ocular Surface Pain: A Narrative Review. Ophthalmol Ther. 2020 September; 9(3):1-21. doi:10.1007/s40123-020-00263-9. Epub 2020 Jun. 5. PMID:32500435; PMCID:PMC7406607.
16. Thompson V, Moshirfar M, Clinch T, Scoper S, Linn S H, McIntosh A, Li Y, Eaton M, Ferriere M, Stasi K. Topical Ocular TRPV1 Antagonist SAF312 (Libvatrep) for Postoperative Pain After Photorefractive Keratectomy. Transl Vis Sci Technol. 2023 Mar. 1; 12(3):7. doi:10.1167/tvst.12.3.7. PMID:36917119; PMCID:PMC10020951.

We claim:

1. Use of a formulation for treating nociceptive and/or neuropathic ocular pain in a subject comprising:
topically administering an effective amount of a formulation comprising a selective Nav1.8 inhibitor to an ocular surface of a subject suffering from nociceptive and/or neuropathic ocular pain, wherein the Nav1.8 inhibitor is a peripheral nerve desensitizing agent, and wherein the Nav1.8 inhibitor is ambroxol and/or a derivative of ambroxol.

2. The use of claim 1, wherein administration of the formulation provides partial preservation of sensation in the eye.

3. The use of claim 2, wherein partial preservation of sensation in the eye comprises a preservation of at least 10% of corneal sensation.

4. The use of claim 2, wherein partial preservation of sensation in the eye comprises a preservation of at least 20% of corneal sensation.

5. The use of claim 1, wherein the ambroxol and/or a derivative of ambroxol is in a weight percent of between 0.001% and 10%, based on total weight or total volume of the formulation.

6. The use of claim 1, wherein the ambroxol and/or a derivative of ambroxol is in a weight percent of between 0.05% and 2.0%, based on total weight or total volume of the formulation.

7. The use of claim 1, wherein the formulation further comprises a cyclodextrin.

8. The use of claim 1, wherein an administration of the formulation is associated with eye trauma or eye surgery.

9. The use of claim 1, further comprising administering an analgesic.

10. The use of claim 9, wherein the formulation is topically administered to the ocular surface before the analgesic is administered.

11. The use of claim 9, wherein the formulation is topically administered at approximately the same time that the analgesic is administered.

12. The use of claim 9, wherein the formulation is topically administered after the analgesic is administered.

13. The use of claim 9, wherein the formulation is topically administered before the onset of postoperative pain or eye trauma pain, wherein the onset of postoperative pain or eye trauma pain has been delayed by the administration of the analgesic.

14. The use of claim 9, wherein the formulation continues to treat nociceptive and neuropathic ocular pain after the effect of the analgesic no longer effective.

15. The use of claim 9, wherein the analgesic comprises tetracaine, proparacaine, chloroprocaine, lidocaine, or bupivacaine.

16. The use of any of claim 9, wherein the analgesic comprises an opioid.

17. The use of any of claim 9, wherein the analgesic comprises a non-steroidal anti-inflammatory drug.

18. The use of claim 1, wherein the formulation is administered within 60 minutes of the subject experiencing eye trauma or eye surgery.

19. The use of claim 1, wherein the formulation is administered within 20 minutes of the subject experiencing eye trauma or eye surgery.

20. The use of claim 1, wherein the formulation is administered within 5 minutes of the subject experiencing eye trauma or eye surgery.

21. The use of claim 1, wherein a device provides the application of the formulation.

22. The use of claim 1, wherein the formulation includes a biocompatible polymer.

23. The use of claim 22, wherein the ambroxol and/or a chemical derivative of ambroxol is impregnated within the biocompatible polymer.

24. The use of claim 22, wherein the biocompatible polymer is dissolved in a carrier.

25. The use of claim 22, wherein the biocompatible polymer comprises poly-2-hydroxyethylmethacrylate (p-HEMA hydrogels), poly(lactic-co-glycolic) acid (PLGA). polycaprolactone (PCL), hydroxypropyl cellulose, Anecortave acetate (AnA) gelatin, and/or collagen.

26. The use of claim 1, wherein the formulation further comprises an additive, wherein the additive is a demulcent, preservative, emollient, or pH-adjusting agent.

27. The use of claim 1, wherein the administration of the effective amount of the formulation results in a decrease in subjective symptoms or the increase of quality-of-life measure.

28. Use of a formulation of claim 1 in the manufacture of a medicament for the treatment of nociceptive and/or neuropathic ocular pain in a subject, wherein the treatment comprises topically administering an effective amount of the formulation to an eye of the subject.

29. A formulation for the use of claim 1 comprising a composition, the composition comprising:
ambroxol and/or a derivative of ambroxol in a weight percent of between 0.01% and 10%, based on total weight or total volume of the formulation; and a cyclodextrin.
